(19) United States
(12) Patent Application Publication
(10) Pub. No.: US 2023/0149329 A1
(43) Pub. Date: May 18, 2023

(54) C5 KETONE COMPOSITIONS AND RELATED METHODS FOR THERAPEUTIC AND PERFORMANCE SUPPLEMENTATION

(71) Applicant: Keto Innovations, LLC, Melissa, TX (US)

(72) Inventors: Ryan P. Lowery, Tampa, FL (US); Jacob Wilson, Tampa, FL (US); Shawn Wells, Frisco, TX (US); Kylin Liao, Frisco, TX (US)

(73) Assignee: Keto Innovations, LLC, Melissa, TX (US)

(21) Appl. No.: 18/157,788

(22) Filed: Jan. 20, 2023

Related U.S. Application Data
(63) Continuation-in-part of application No. 17/732,482, filed on Apr. 28, 2022.
Application No. 17/732,482 is a continuation-in-part of application No. 16/306,412, filed on Nov. 30, 2018, now Pat. No. 11,337,945.
(60) Provisional application No. 62/343,941, filed on Jun. 1, 2016.

(51) Int. Cl. A61K 31/19; A61P 25/28; A61P 3/00

(52) U.S. Cl. CPC A61K 31/19; A61P 25/28; A61P 3/00

(57) ABSTRACT

The present disclosure pertains to compositions and methods for the treatment and/or prevention of one or more of obesity, diabetes, metabolic syndrome, Alzheimer's disease, Chronic Fatigue Syndrome (CFS), aging, fibromyalgia, dyslipidemia, hypercholesterolemia, dyslipidemia, Parkinson's disease, migraines, Traumatic Brain Injury (TBI), Attention Deficit Disorder (ADD)/Attention Deficit Hyperactivity Disorder (ADHD), Cancer, Cardiovascular Disease (CVD)/Coronary Artery Disease (CAD), Chronic Pain, neuralgia, depression, amyotrophic lateral sclerosis (ALS), and epilepsy, Insufficient Cellular Energy (ICE) and mitochondrial dysfunction. The present disclosure also pertains to methods for increasing mental and/or physical performance levels and/or decreasing exertion during exercise in a subject by the administration of C5 ketones.

### CROSS-REFERENCE TO RELATED APPLICATIONS

This application is a continuation in part of U.S. patent application Ser. No. 17/732,482 which was filed on Apr. 28, 2022, and is entitled “C5 KETONE COMPOSITIONS, AND RELATED METHODS, FOR THERAPEUTIC AND PERFORMANCE SUPPLEMENTATION”, which is a continuation in part of U.S. patent application Ser. No. 16/306,412 which was filed on Nov. 30, 2018 and is entitled “C5 KETONE COMPOSITIONS, AND RELATED METHODS, FOR THERAPEUTIC AND PERFORMANCE SUPPLEMENTATION”, which is a U.S. National Stage of International Application No. PCT/IB2017/054679, filed Aug. 1, 2017, which designates the U.S., published in English, and claims the benefit of U.S. Provisional Application Ser. No. 62/343,941, filed Jun. 1, 2016, all of which are hereby incorporated by reference for all purposes.

### BACKGROUND

Beta-hydroxybutyrate (BHB) (a C4 ketone) and its salts, esters, and combinations thereof have become increasingly common in the marketplace for the ability to increase blood BHB levels, promote increases in cognitive function, and an array of benefits that may benefit diseased individuals.

The heart, brain and kidney are the three organs with the highest activity of enzymes utilizing ketone bodies (Ardawi and Newsholme 1982). For instance, studies have shown an improvement in the neurological status of a newborn with pyruvate carboxylase deficiency during treatment with triheptanoin (C7 ketone) (Mochel et al 2005; Roe and Mochel 2006).

### SUMMARY

In various implementations, a therapeutically effective amount of one or more C5 ketone body, pharmaceutically acceptable salt, ester, chelate thereof, and/or combinations thereof may be administered to increase mental and/or physical performance levels in a subject. Administration may increase and/or maintain ketosis, increase blood ketone levels, increase and/or maintain weight loss, increase fat loss, improve body composition, increase strength (e.g., power, muscle mass), increase energy, and/or increase cognitive health (e.g., cognitive function, memory, focus, motor function associated with age or disease related declines, etc.), glucose tolerance, maintain and/or reduce blood glucose levels, reduce or inhibit progression of symptoms of traumatic brain injury, and/or reduce or inhibit progression of cognitive decline symptoms. The pharmaceutically effective amount of C-5 ketone bodies may be administered to healthy and/or not healthy (e.g., with diseases such as obesity, etc. and/or disorders) individuals.

In some implementations, the methods described aid Krebs cycle function and/or anaplerosis in a subject being treated with the described compositions. In some implementations, the C5 ketone body is beta-ketopentanoate (β-ketopentanoate) and/or beta-hydroxypentanoate (β-hydroxypentanoate).

In some implementations, the C5 ketone body may be administered with one or more additional compounds (β-hydroxybutyrates, fatty acids, triglycerides, vitamins, minerals, amino acids, proteins, etc.). The C5 ketone body may be administered with one or more pharmaceutically acceptable binders and/or carriers.

The compositions that include the C5 ketone body may be administered to treat of the diseases, conditions, and/or symptoms thereof as described herein. In various implementations, approximately 1 to approximately 50 g of the C5 ketone body, pharmaceutically acceptable salt, ester, chelate thereof, and/or combinations thereof may be administered (e.g., orally) to an individual.

The details of one or more implementations are set forth in the accompanying drawings and the description below. Other features, objects, and advantages of the implementations will be apparent from the description and drawings.

### BRIEF DESCRIPTION OF DRAWINGS

For a more complete understanding of this disclosure and its features, reference is now made to the following description, taken in conjunction with the accompanying drawings, in which:

FIG. 1 illustrates the metabolism of C5 ketones in their racemic mixture and dissociated into their respective isoforms.

FIG. 2 illustrates C4 ketogenesis and C5 ketogenesis in the liver. Numbers refer to the following enzymes: 3-ketoacyl-CoA thiolase (1); HMG-CoA synthase (2); HMG-CoA lyase (3); and β-hydroxybutyrate dehydrogenase(4). The figure also shows the link between propionyl-CoA and the CAC via anaplerosis.

FIG. 3 illustrates the metabolism of triheptanoin into C5 ketone bodies β-hydroxypentanoate (BHP) and β-ketopentanoate (BKP), which ultimately get transported out of the mitochondria.

FIG. 4 shows a graph of blood glucose level with BKP administration.

FIG. 5 shows a graph of blood ketone level with BHP administration.

FIG. 6 shows a graph of visual analog scales (VAS) of well-being with BKP administration.

FIG. 7 shows a graph of VAS of energy with BKP administration.

FIG. 8 shows a graph of VAS of focus with BKP administration.

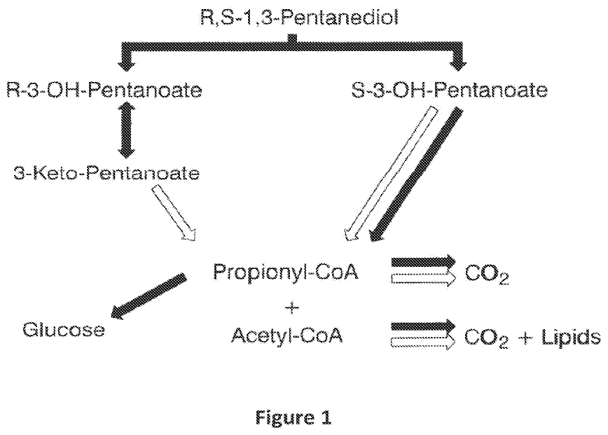

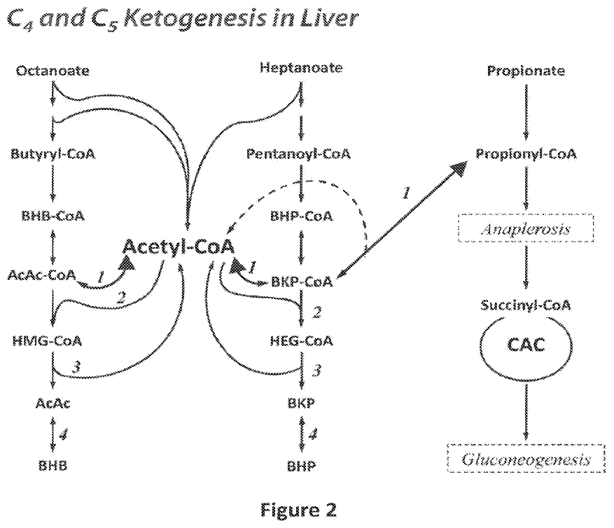

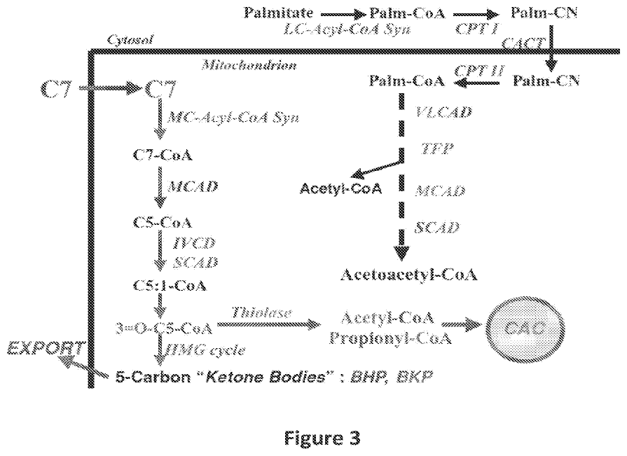

Like elements in the various figures are denoted by like reference numerals for consistency.

### DETAILED DESCRIPTION

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular implementations only and is not intended to be limiting.

As used herein, the indefinite articles “a”, “an” and “the” should be understood to include plural reference unless the context clearly indicates otherwise. As used in this specification, the singular forms “a”, “an” and “the” include plural referents unless the content clearly indicates otherwise. Thus, for example, reference to “a compound” includes a combination of two or more compounds and reference to “a C5 ketone body” includes different types and/or combinations of C5 ketone bodies. As another nonlimiting example, reference to “C5 ketone bodies” may include a single type of C5 ketone body. As another nonlimiting example, reference to “a composition” includes a combination of two or more compositions.

The terms “approximately”, “about”, and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as defined above for “and/or”. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “subject” refers to an animal Typically, the terms “subject” and “patient” may be used interchangeably herein in reference to a subject. As such, a “subject” includes an animal that is being treated for conditions described in the present disclosure, or the recipient of a mixture of components or compositions as described herein. The term “animal,” includes, but is not limited to, a mammal, such as a mouse, rat, dog, guinea pig, cow, horse, chicken, cat, rabbit, pig, monkey, chimpanzee, and human.

The term “effective amount,” “pharmaceutically effective amount,” or “therapeutically effective amount,” as used herein, means that amount of active compound that elicits the biological or medicinal response in a tissue system, animal or human that is being sought by a researcher, veterinarian, medical doctor or other clinician, which includes alleviation of one or more of the symptoms of the disease or disorder being treated; reduction of the severity of one or more of the symptoms of the disease or disorder being treated; or otherwise provides the desired effect. The precise dosage will vary according to a variety of factors such as subject-dependent variables (e.g., age, medical history, etc.), the disorder or condition, and the treatment being affected.

The term “pharmaceutically acceptable,” as used herein, means approved by a regulatory agency of the Federal or a state government or listed in the U.S. Pharmacopeia or other generally recognized pharmacopeia for use in animals and/or in humans. “Pharmaceutically acceptable salt” refers to a salt of a compound of the disclosure that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethane sulfonic acid, 1,2-ethane-disulfonic acid, 2-hydroxyethanesulfonic acid, benzene sulfonic acid, 4-chlorobenzenesulfonic acid, 2naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced.

The term “carrier,” as used herein, refers to a diluent, adjuvant, excipient, and/or vehicle with which the compositions are administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, sucrose, gelatin, lactose, malt, rice, flour, chalk, silica gel, sodium stearate, glycerol monostearate, talc, sodium chloride, glycerol, propylene, glycol, water, ethanol, and the like. The pharmaceutical composition(s) may also contain wetting or emulsifying agents or suspending/diluting agents, or pH buffering agents, or agents for modifying or maintaining the rate of release of the formulations. The compositions can take the form of solutions, suspensions, emulsion, tablets, pills, capsules, powders, gels, creams, sustained-release formulations, and the like. Formulations can include standard carriers such as pharmaceutical grades of mannitol, lactose, sodium saccharine, starch, magnesium stearate, cellulose, magnesium carbonate, etc. Such compositions will contain an effective amount of the active ingredient(s) together with a suitable amount of carrier so as to provide the proper form to the subject based on the mode of administration to be used.

“Preventing” or “prevention,” as used herein, refers to a reduction in risk of acquiring disorder (i.e., causing at least one of the clinical symptoms of the disease/disorder not to develop in a subject that may be predisposed to the disorder but does not yet experience or display symptoms of the disorder).

The term “treating”, or “treatment” of any disorder refers, in one implementation, to ameliorating the disorder (i.e., arresting or reducing the development of the disorder (e.g., Alzheimer's disease) or at least one of the clinical symptoms thereof). In another implementation “treating” or “treatment” refers to ameliorating at least one physical parameter, which may not be discernible by the subject. In yet another implementation, “treating” or “treatment” refers to modulating the disorder, either physically, (e.g., stabilization of a discernible symptom), physiologically, (e.g., stabilization of a physical parameter), or both. In yet another implementation, “treating” or “treatment” refers to delaying the onset of the disorder.

In various implementations, a composition that includes one or more C5 ketone bodies may be administered to a subject in a therapeutically effective amount. The composition may be orally administered. The composition may include one or more additional compounds.

C5 ketone bodies may be naturally formed in the liver from the partial oxidation of odd chain fatty acids (e.g., C-7, heaptanoic acid, like macadamia nut oil). C5 ketones have been shown to occur in trace amounts in human plasma and most plants. C5 ketone bodies are derived from the oxidation in liver of the (R) enantiomer of R, S-1,3-pentanediol, a potential nutrient. Whether administered by bolus or constant infusion, the two substrates are interconverted and rapidly metabolized in an animal model.

During short-term experiments, 3-hydroxypentanoate (BHP) and β-ketopentanoate (BKP) are well metabolized in animals without apparent intolerance to a large supply of propionyl-CoA. In nondiabetic animals, C5 ketone bodies ((R)-B-hydroxypentanoate and β-ketopentanoate) are very rapidly used when administered intravenously at doses corresponding to 75% of the energy requirement (Leclerc et al 1995).

C5 ketones have potential to work directly on the mitochondria, therefore, in some implementations, the C5 ketones and related methods described by this disclosure are useful for treating mitochondrial dysfunction. As such, C5 ketones are particularly useful in treating cancers that result from mitochondrial dysfunction, such as, for example, glioblastoma.

In general, the embodiments are directed to compositions and methods for increasing mental and/or physical performance levels in a subject. Administration may increase and/or maintain ketosis, increase blood ketone levels, increase and/or maintain weight loss, increase fat loss, improve body composition, increase strength (e.g., power, muscle mass), increase energy, and/or increase cognitive health (e.g., cognitive function, memory, focus, motor function associated with age or disease related declines, etc.), glucose tolerance, maintain and/or reduce blood glucose levels, reduce or inhibit progression of symptoms of traumatic brain injury, and/or reduce or inhibit progression of cognitive decline symptoms.

In various implementations, the embodiments may be utilized for the treatment and/or prevention of one or more of obesity; diabetes; metabolic syndrome; Alzheimer's disease; Chronic Fatigue Syndrome (CFS); aging; fibromyalgia; dyslipidemia; hypercholesterolemia; dyslipidemia; Parkinson's disease; migraines; Traumatic Brain Injury (TBI); Attention Deficit Disorder (ADD)/Attention Deficit Hyperactivity Disorder (ADHD); Cancer, such as for example, glioblastoma; Cardiovascular Disease (CVD)/Coronary Artery Disease (CAD); Chronic Pain; neuralgia; depression; amyotrophic lateral sclerosis (ALS); epilepsy; Insufficient Cellular Energy (ICE); mitochondrial dysfunction; and/or combinations thereof.

The embodiments described herein may be administered for healthy and/or not healthy (e.g., with diseases such as obesity, etc. and/or disorders) individuals. Administration in an individual may promote and/or maintain ketosis, cause weight loss and/or manage weight, and/or increase blood ketone levels. The administration may cause weight loss and/or maintenance; elevated β-hydroxybutyrate levels in the blood; elevated, reduced, and/or maintenance of blood ketone levels; induction and/or maintenance of ketosis; and/or reduction; improve mental acuity; improve focus; improve energy; improve cognitive function; reduce traumatic brain injury; improve diabetes; improve glucose tolerance; decrease blood glucose levels; reduce neurological disorders and/or symptoms thereof; improve cancer and/or symptoms thereof; improve inflammatory conditions; suppressing appetite; improve symptoms associated with aging; provide anti-glycation affects; improve epilepsy and/or symptoms thereof; improve depression and/or symptoms thereof; improve performance; improve strength; increase muscle mass; increase fat loss; improve body composition; improve energy; improve focus; improve cognitive function; improve mood and/or well-being; and/or combinations thereof.

In general, the embodiments are directed to compositions and methods for increasing blood ketone levels in a subject, the method comprising administering to the subject a therapeutically effective amount of a five carbon (C5)-ketone body, or pharmaceutically acceptable free acid, salt, ester, or chelate thereof.

In general, the embodiments are directed to compositions and methods decreasing blood glucose levels in a subject, the method comprising administering to the subject a therapeutically effective amount of a five carbon (C5)-ketone body, or pharmaceutically acceptable free acid, salt, ester, or chelate thereof.

In general, the embodiments are directed to compositions and methods aiding in weight loss in a subject, the method comprising administering to the subject a therapeutically effective amount of a five carbon (C5)-ketone body, or pharmaceutically acceptable free acid, salt, ester, or chelate thereof.

In general, the embodiments are directed to compositions and methods for increasing mental and/or physical performance levels, the method comprising administering to the subject a therapeutically effective amount of a five carbon (C5)-ketone body, or pharmaceutically acceptable free acid, salt, ester, or chelate thereof. The mental performance level being increased may be a measure of well-being and/or focus. The physical performance level being increased may be a measure of energy.

In general, the embodiments are directed to compositions and methods for lowering exertion during exercise in a subject, the method comprising administering to the subject a therapeutically effective amount of a five carbon (C5)-ketone body, or pharmaceutically acceptable free acid, salt, ester, or chelate thereof. In some implementations, the exercise is moderate intensity exercise and in other implementations, the exercise is high intensity exercise. Moderate intensity exercise includes, for example, running, cycling, rowing, circuit training, and yoga. High intensity exercise includes, for example, sprinting, hockey, soccer, baseball, football, basketball, resistance training and boxing.

In various embodiments, a composition is provided that includes at least one five carbon (C5) ketone body. The composition may be administered to a subject in a therapeutically effective amount.

In various embodiments, the composition may include approximately 100 mg to approximately 50 g of one or more C5 ketone bodies. The C5 ketone bodies of the composition may include one or more C5 ketone bodies such as β-hydroxypentanoate and/or β-ketopentanoate.

In some implementations, the compositions comprising one or more C5 ketone body, or pharmaceutically acceptable salt, ester, or chelate thereof, as the active ingredient. The compositions described can also be in the form of a pharmaceutical composition comprising one or more pharmaceutically acceptable carriers described herein. The compounds and compositions of the disclosure can be used to treat, alleviate, inhibit, or prevent the diseases and conditions further described herein. Further, the compounds and compositions of the disclosure can be used for increasing mental and/or physical performance levels in a subject or for aiding in weight loss. In some implementations, the C5 ketone body, or pharmaceutically acceptable salt, ester, or chelate thereof, concentration is from about 1 g to about 50 g, from about 1 g to about 40 g, from about 1 g to about 30 g, from about 1 g to about 20 g, from about 1 g to about 10 g. In some implementations, the C5 ketone, or pharmaceutically acceptable salt, ester, or chelate thereof, concentration is about 10 g, from about 10 g to about 50 g, from about 20 g to about 50 g, from about 30 g to about 50 g, from about 40 g to about 50 g.

The composition may include C5 ketone bodies in any appropriate therapeutically effective concentration, which can be expressed for example, as a weight percent, molarity, molality, etc. For example, in some implementations, compositions may contain amounts of C5 ketone bodies, by wt. %, that are more than about 0%, more than about 10%, more than about 20%, more than about 30%, more than about 40%, more than about 50%, more than about 60%, more than about 70%, more than about 80%, more than about 90%. For example, in some implementations, compositions may contain amounts of C5 ketone bodies, by wt. %, that are less than about 100%, less than about 90%, less than about 80%, less than about 70%, less than about 60%, less than about 50%, less than about 40%, less than about 30%, less than about 20%, less than about 10%.

In various embodiments, any appropriate C5 ketone body may be utilized. For example, C5 ketone body may include β-hydroxypentanoate (BHP) (also known as 3-hydroxypentanoic acid, 3-Hydroxyvalerate, 3-Hydroxy valeric acid, and β-hydroxyvaleric acid) and/or β-ketopentanoate (BKP) (also known as 3-Oxopentanoic acid, 3-Oxopentanoate 3-Oxovaleric acid, and 3-Ketovaleric acid).

In various implementations, a composition including a pharmaceutically effective amount of the C5 ketone bodies may be administered to an individual to promote and/or maintain ketosis, cause weight loss and/or manage weight, and/or increase blood ketone levels. For example, approximately 0.1 g to approximately 50 g of BHP and/or BKP may be administered to a subject. In some implementations, approximately 0.1 g to approximately 15 g of BHP and/or BKP may be administered to an individual. In some implementations, approximately 1 g to approximately 10 g of BHP and/or BKP may be administered, for example, once a day to 5 times a day. In some implementations, less than 3 g of C5 ketone bodies may be administered to the subject (e.g., approximately 0.1 to approximately 3 g of BHP and/or BKP).

In one or more embodiments, the C5 ketone body can be provided as one or more stereoisomers, also sometimes referred to as enantiomers, diastereomers, or diastereoisomers. The stereoisomers can be differentiated based on a directional of rotation of plane-polarized light (L/D), or an absolute configuration of the chiral centers (R/S). For example, a composition containing β-hydroxypentanoate may comprises at least one stereoisomer selected from (R)-β-hydroxypentanoate, and (S)-β-hydroxypentanoate. A composition containing β-ketopentanoate may comprises at least one stereoisomer selected from (R)-β-ketopentanoate, and (S)-β-ketopentanoate.

In some implementations, the C5 ketone bodies may include racemic mixtures and/or enriched mixtures (e.g., one form may be predominant) to produce a predetermined effect during administration. For example, the C5 ketones utilized in the methods and compositions may be provided in an isolated or purified form. Synthetic variants of the C5 ketones may be utilized, either individually or in combination with other synthetic C5 ketone variants, or in combination with non-synthetic variants.

For example, in some embodiments, the C5 ketone body may comprise a racemic mixture of β-hydroxypentanoate containing about 50% by equivalents of (R)-β-hydroxypentanoate and about 50% by equivalents of (S)-β-hydroxypentanoate. In some embodiments, the C5 ketone body may comprise a racemic mixture of β-ketopentanoate containing about 50% by equivalents of (R)-β-ketopentanoate and about 50% by equivalents of (S)-β-ketopentanoate.

In some embodiments, the composition may comprise a non-racemic mixture containing more than 0% and less than 100% by equivalents of at least one of the β-hydroxypentanoate stereoisomer. In some embodiments, the composition may comprise a non-racemic mixture containing more than 0% and less than 100% by equivalents of at least one of the β-ketopentanoate stereoisomer.

In one or more embodiments, a non-racemic mixture may contain more of the (R)-β-hydroxypentanoate or (R)-β-ketopentanoate enantiomers relative to (S)-β-hydroxypentanoate or (S)-β-ketopentanoate enantiomers. Compositions containing more of the (R) enantiomers may provide a greater, faster and/or prolonged ketogenic effect compared to a racemic mixture. Administering the (R) enantiomers to a subject may provide an additional quantity and/or increased blood plasma level that can be immediately utilized by the body, such as for conversion to propionyl-CoA. The presence of the S-enantiomer may modulate and extend this effect.

For example, in one or more embodiments, the composition may comprise a non-racemic mixture containing more than 50%, more than 60%, more than 70%, more than 80%, more than 90%, and less than 100% by equivalents of (R)-β-hydroxypentanoate. The composition may additionally comprise less than 50%, less than 40%, less than 30%, less than 20%, less than 10%, and more than 0% by equivalents of (S)-β-hydroxypentanoate.

The composition may comprise a non-racemic mixture containing an advantageous ratio between stereo isomers. For example, the composition may contain (R)-β-hydroxypentanoate and at least one other stereoisomer, wherein the stereoisomer ratio of (R)-β-hydroxypentanoate to the at least one other diastereomer is at least 2:1, at least 3:1, at least 4:1.

For example, in one or more embodiments, the composition may comprise a non-racemic mixture containing more than 50%, more than 60%, more than 70%, more than 80%, more than 90%, and less than 100% by equivalents of (R)-β-ketopentanoate. The composition may additionally comprise less than 50%, less than 40%, less than 30%, less than 20%, less than 10%, and more than 0% by equivalents of (S)-β-ketopentanoate.

The composition may comprise a non-racemic mixture containing an advantageous ratio between stereo isomers. For example, the composition may contain (R)-β-ketopentanoate and at least one other stereoisomer, wherein the stereoisomer ratio of (R)-β-ketopentanoate to the at least one other diastereomer is at least 2:1, at least 3:1, at least 4:1.

When administered, the different diastereomers of the non-racemic compositions may provide one or more benefits. For example, (R)-β-hydroxypentanoate and/or (R)-β-ketopentanoate may be interconverted by mitochondrial BHB dehydrogenase more readily than other diastereomers. In peripheral tissues, especially heart, kidney, and brain which have high activities of 3-oxoacid-CoA transferase, (R)-β-hydroxypentanoate and/or (R)-β-ketopentanoate may be at least partially converted to propionyl-CoA (which is anaplerotic), aiding Krebs cycle function.

In one or more embodiments, a non-racemic mixture may contain more of the (S)-β-hydroxypentanoate or (S)-β-ketopentanoate enantiomers relative to (R)-β-hydroxypentanoate or (R)-β-ketopentanoate enantiomers. The presence of the (S)-enantiomer may modulate and extend the ketogenic effect provided by the (R) enantiomers. Thus, compositions containing more of the (S) enantiomers may provide a prolonged ketogenic effect compared to a racemic mixture.

For example, in one or more embodiments, the composition may comprise a non-racemic mixture containing more than 50%, more than 60%, more than 70%, more than 80%, more than 90%, and less than 100% by equivalents of (S)-β-hydroxypentanoate. The composition may additionally comprise less than 50%, less than 40%, less than 30%, less than 20%, less than 10%, and more than 0% by equivalents of (R)-β-hydroxypentanoate.

The composition may comprise a non-racemic mixture containing an advantageous ratio between stereo isomers. For example, the composition may contain (S)-β-hydroxypentanoate and at least one other stereoisomer, wherein the stereoisomer ratio of (S)-β-hydroxypentanoate to the at least one other diastereomer is at least 2:1, at least 3:1, at least 4:1.

For example, in one or more embodiments, the composition may comprise a non-racemic mixture containing more than 50%, more than 60%, more than 70%, more than 80%, more than 90%, and less than 100% by equivalents of (S)-β-ketopentanoate. The composition may additionally comprise less than 50%, less than 40%, less than 30%, less than 20%, less than 10%, and more than 0% by equivalents of (R)-β-ketopentanoate

The composition may comprise a non-racemic mixture containing an advantageous ratio between stereo isomers. For example, the composition may contain (S)-β-ketopentanoate and at least one other stereoisomer, wherein the stereoisomer ratio of (S)-β-ketopentanoate to the at least one other diastereomer is at least 2:1, at least 3:1, at least 4:1.

Administration of (S)-β-hydroxypentanoate may result in increased endogenous production of (R)-β-hydroxypentanoate, and endogenous conversion into (R)-β-hydroxypentanoate, fatty acids and sterols, promoting prolonged ketosis. Administration of (S)-β-hydroxypentanoate may also result in ketone metabolism independent of conversion to (R)-β-hydroxypentanoate.

Administration of (S)-β-ketopentanoate may result in increased endogenous production of (R)-β-ketopentanoate, and endogenous conversion into (R)-β-ketopentanoate, fatty acids and sterols, promoting prolonged ketosis. Administration of (S)-β-ketopentanoate may also result in ketone metabolism independent of conversion to (R)-β-ketopentanoate.

In some embodiments, the C5 ketone bodies may include free ketone bodies, salts, esters, chelates, complexes, other appropriate forms, and/or combinations thereof of BKP and/or BHP. For example, C5 ketone bodies may include C5 ketone salts (e.g., sodium, calcium, magnesium, etc.), esters (e.g., glycerol), chelates (e.g., amino acids), as well as C5 ketone bodies that are unbound in their “free acid” state, or any other appropriate chemical variant with C5 ketones attached.

In various implementations, the C5 ketone bodies may include one or more of the described components, equivalent(s) of the described component(s), derivatives of the described component(s), complex(es) of the described component(s), salt(s) of the described component(s), and/or combinations thereof.

For example, the β-hydroxypentanoate may be characterized by the following chemical structure:

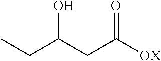

wherein X is a hydrogen, a metal ion, an amino cation, an amino acid cation, an alkane, an alkenyl, or an aryl.

For example, the β-ketopentanoate may be characterized by the following chemical structure:

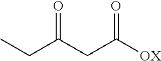

wherein X is a hydrogen, a metal ion, an amino cation, an amino acid cation, an alkane, an alkenyl, or an aryl.

In some implementations, the composition may include one or more C5 ketone body salts. The cation associated with the C5 ketone body salts may be any appropriate cation such as sodium, potassium, magnesium, and calcium, and transition metals such as zinc, selenium, chromium, and iron, as well as other appropriate minerals, and/or combinations thereof.

The salt cations may be chosen to produce a specific effect in the body. For example, one or more of the cations in the composition may operate in a similar manner as an electrolyte drink (e.g., by utilizing a predetermined amount of sodium, potassium, calcium, and/or magnesium).

The C5 ketone body salts may be provided as a combination of salts. For example, the C5 ketone body salts may be a combination of at least two salts, a combination of at least three salts, or a combination of at least four salts.

In some implementations, the composition may include C5 ketone body salt and one other slower blood ketone elevating C5 ketone body. For example, an individual may be administered a first weight amount of BHP salt and/or BKP salt and a second weight amount of ester of C5 ketone body and/or a polymer of C5 ketone body. The first amount and the second amount may be different or the same.

In some implementations, two or more, three or more, and/or four or more different C5 ketone body salts may be selected to produce a predetermined effect in the body (e.g., high initial spike in blood ketone levels, approximately even increase in blood ketone levels for a predetermined period of time such as 60 and/or 90 minutes, etc., a combination with low magnesium to reduce gastrointestinal issues, a combination with relatively higher magnesium to facilitate sleep while maintaining composition administration schedule(s), etc.).

In some embodiments, the composition may include and/or may be administered with, and/or coupled to a compound such as an amino acid or amino acid metabolite. For example, the composition may include one or more amino acids such as leucine, iso-leucine, taurine, purine, lysine, arginine, histidine, ornithine, creatine, glutamine, agmatine, citrulline and/or combinations thereof. Administration of amino acids with C5 ketone bodies may reduce the intake of cations associated with salts (e.g., which may inhibit side effects associated with administration) and/or allow administration of another compound that has health benefits (e.g., administration of some amino acid may promote smooth muscle growth and/or cell repair).

In some implementations, approximately 0.5 g to approximately 10 g of amino acid may be administered with one or more C5 ketone bodies. For example, less than approximately 50 g of C5 ketone body may be administered with less than approximately 60 mg of an amino acid, such as leucine, may be administered daily. In some implementations, approximately 0.5 g to approximately 2 g of an amino acid, such as leucine, may be administered with a C5 ketone body. For example, approximately the composition administered may include approximately 0.1 to approximately 7 g C5 ketone body and approximately 1-3 g of leucine. The C5 ketone body and the leucine may be a mixture; administered separately and proximate in timing; a complex, and/or administered in any other appropriate manner.

In some embodiments, the composition may include one or more esters of β-hydroxypentanoate. For example, one or more of the C5 ketone bodies may include ester derivatives including alkyl ester derivatives, such as methyl, ethyl, propyl, butyl, hexyl, etc. Ester derivatives may include as one or more esters, such as mono-, di-, tri-, oligo-, and/or polyesters. For example, the C5 ketone bodies may include esters such as mono-esters of ethanol, mono-ester of 1-propanol, mono-ester of 1,2-propanediol, di-ester of 1,2-propanediol, mono-ester of 1,3-propanediol, di-ester of 1,3-propanediol, mono-ester of S—, R—, or S—R-1,3-butanediol, di-ester of S—, R—, or S—R-1,3-butanediol, mono-ester of glycerin, mono-ester, di-ester of glycerin, tri-ester of glycerin, and/or combinations thereof.

In some embodiments, the composition may include one or more stable free acids of the C5 ketone body. For example, the C5 ketone body may comprise β-hydroxypentanoic acid, β-ketopentanoic acid, and combinations thereof.

In some implementations, the composition may include isomers and/or polymers of C5 ketone bodies, as appropriate. the composition may include a long-acting component and/or be long-acting. For example, since the body digests polymers and/or esters of C5, the delivery of C5 ketone bodies may be slower than a digestion of a C5 ketone body salt. In some implementations, the composition may include a C5 ketone body salt and a long-acting C5 ketone body form (e.g., polymer, ester, coated and/or processed form to provide slow release).

For example, in some implementations, polymers of the C5 ketone bodies may include one or more ingestible, bio-derived and/or biodegradable hydroxyalkanoate polymers or copolymers, formed from 5 carbon monomer units, such as polyhydroxypentanoate (PHP), polyketopentanoate (PKP) and/or copolymers of PHP and/or PKP. the five carbon monomer units may be copolymerized with monomer units of having different carbon lengths, such as one or more C4 ketone bodies.

In some embodiments, the composition may include one or more additional compounds that may or may not be capable of independently increasing ketone levels, maintaining ketone levels, inducing ketosis, and/or maintaining ketosis. For example, additional compounds capable of independently increasing blood ketone levels may include short chain fatty acids (e.g., fatty acid with between 2 carbons than 6 carbons), short chain triglycerides (e.g., triglycerides with less than 6 carbons), medium chain fatty acids (e.g., fatty acid with 6-12 carbons), medium chain triglycerides (e.g., triglycerides with 7-12 carbons), long chain fatty acids (e.g., fatty acids with more than 12 carbons), long chain triglycerides (e.g., triglycerides with more than 12 carbons), and/or combinations thereof.

As referred to herein, a short chain fatty acid, or the mono-, di- or triglyceride of the short-chain fatty acid, has a carbon chain length of less than 6 carbons. The short chain fatty acids and/or triglycerides may include, for example but not limited to, acetate, propionate, and/or butyrate.

For example, the composition may include a short chain compound (e.g., short chain fatty acid and/or short chain triglyceride) that may unexpectedly increase ketone concentrations in the blood more than the administration of similar amounts of C5 ketone bodies and medium chain compounds (e.g., short chain fatty acid and/or short chain triglyceride) and/or may increase ketone concentrations in the blood more than each component individually.

As referred to herein, a medium chain fatty acid, or the mono-, di- or triglyceride of the medium-chain fatty acid, has a carbon chain length of from 6 to 12 carbons. The medium chain fatty acids and/or triglycerides may include for example but not limited to, lauric acid and/or coconut oil, coconut milk powder, fractionated coconut oil, isolated hexanoic acid, isolated octanoic acid, isolated decanoic acid, ethoxylated triglyceride, triglyceride derivatives thereof, aldehyde triglyceride derivatives thereof, monoglyceride derivatives thereof, diglyceride derivatives thereof, triglyceride derivatives thereof, and/or alkyl esters thereof.

As referred to herein, a long chain fatty acid, or the mono-, di- or triglyceride of the long-chain fatty acid, has a carbon chain length of more than 12 carbons. Long chain fatty acids and/or triglycerides may include for example but not limited to, dairy products and/or palm oil.

In some implementations, the composition may include one more citrates. The citrate may be provided as an alkali metal salt, such as sodium or potassium, and/or as an alkali earth salt, such as magnesium or calcium. Compositions including one or more citrates may reduce the acidic environment of the body due to circulating ketone bodies. Compositions including one or more citrates may therefore reduce the risk of metabolic acidosis, nephrolithiasis, nephrocalcinosis, and bone mineral density loss.

For example, the composition may additionally contain amounts of citrates, by wt. %, that are more than about 0%, more than about 1%, more than about 2%, more than about 3%, more than about 4%, more than about 5%, more than about 10%, more than about 20%, more than about 30%, more than about 40%, and/or less than about 50%. A unit dosage of the composition may contain amounts of citrates that are more than about 10 mg, more than about 50 mg, more than about 100 mg, more than about 200 mg, more than about 500 mg, and/or less than about 1000 mg.

In some implementations, the composition may include one or more cannabinoid compounds derived from the cannabis plant. The cannabinoid compounds can include cannabidiol (CBD), tetrahydrocannabivarin (THCV), cannabigerol (CBG), cannabidivarin (CBDV), cannabichromene (CBC), cannabinol (CBN), cannabielsoin (CBE), tetrahydrocannabinol (THC), iso-THC, cannabicyclol (CBL), cannabicitran (CBT), cannabivarin (CBV), cannabichromevarin THC (CBCV), cannabigerovarin (CBGV), cannabigerol monomethyl ether (CBGM), a carboxylic acid form of a cannabinoid, and combinations thereof.

For example, the composition may include approximately 0.5 to approximately 50 g of C5 ketone bodies and approximately 5 mg to approximately 300 mg CBD. As another non-limiting example, the composition may include approximately 0.5 to approximately 50 g of C5 ketone bodies, approximately 0.5 to approximately 20 g of β-hydroxybutyrate, and approximately 5 mg to approximately 300 mg CBD. Cannabinoid compounds can be attached to the ketone bodies via, for example, an ester bond.

In some implementations, a pharmaceutically effective amount of composition may include a pharmaceutically effective amount of long chain fatty acid and/or triglyceride. For example, 0.1-50 g of C5 ketone bodies and 0.1 to 50 g of long chain fatty acid may be administered to an individual between 1-5 times a day. In some implementations, approximately 1 g to approximately 3 g of C5 ketone bodies and approximately 1 g of long chain fatty acid and/or triglyceride may be administered from once a day to approximately 5 times a day.

In some implementations, C5 ketone bodies, short chain compound(s) (e.g., fatty acids and/or triglycerides, butyrate), and/or medium chain compound(s) (e.g., fatty acids and/or triglycerides) may be administered approximately simultaneously and/or sequentially to an individual. For example, approximately 0.1 g to approximately 50 g C5 ketone bodies, approximately 0.1 g to approximately 50 g short chain triglyceride, and approximately 0.1 g to approximately 50 g medium chain fatty acid such as lauric acid and/or coconut oil may be administered between 1-5 times a day. In some implementations, approximately 1 g to approximately 3 g of C5 ketone bodies and approximately 1 g of short chain fatty acid and/or triglyceride and/or approximately 1 g of medium chain fatty acid and/or triglyceride may be administered from once a day to approximately 5 times a day. In some implementations, approximately 0.1 g to approximately 20 g C5 ketone bodies (e.g., salts, esters, isomers, and/or other appropriate forms) may be administered in humans.

In some embodiments, the composition may include a fat burner supplement for increasing lipolysis and/or fat oxidation. The fat burner supplements may include, for example but not limited to, green tea, green tea extract, isolated green tea catechins, epigallocatechin gallate (EGCG), green coffee extract, conjugated linoleic acid (CLA), tetradecyl thioacetic acid (TTA), Coleus forskohlii, yohimbine, rauwolscine, capsaicin, raspberry ketones, 4-(4-hydroxyphenyl) butan-2-one, p-hydroxybenzyl acetone, ephedrine, synephrine, octopamine, 1,3-dimethylamylamine, higenamine, fucoxanthin, acetylcholine modulators and/or adenosine receptor antagonists, caffeine, nicotine, coca leaf derivatives, ursolic acid, clenbuterol, noradrenaline reuptake inhibitors, hordenine, atomoxetine, 7-oxodehydroepiandrosterone, triiodothyronine, and combinations thereof.

In some embodiments, the composition may include a nootropic supplement for increasing cognitive performance, alertness, and/or mood. The nootropic compounds may include, for example but not limited to, tyrosine, L-DOPA, tryptophan, 5-hydroxytryptophan (5-HTP), racetams, piracetam, oxiracetam, aniracetam, L-theanine, D-serine, phosphatidylserine, tolcapone, uridine, vinpocetine, norepinephrine reuptake inhibitors, hordenine, atomoxetine, Mucuna pruriens, Panax ginseng, Ginkgo biloba, Rhodiola rosea, Polygala tenuifolia, Muira puama, Huperzia serrata, Eschscholzia californica, Convolvulus pluricaulis, Centella asiatica, Evolvulus alsinoides, Bacopa monnieri, Epimedium herbs, Ashwagandha herbs, cyclic adenosine monophosphate (cAMP) modulators, forskolin, nicotine, caffeine, amphetamines, coca leaf derivatives, cholinergic compounds, acetylcholine modulators, huperzine-A, dimethylaminoethanol, methylliberine, theacrine, phenylalanine, phenylethylamine, citicoline, choline, alpha-glycerophosphocholine, and combinations thereof.

In some embodiments, the composition may include a stimulant component. In some implementations, the composition with a stimulant component may increase mental processes (e.g., acuity including cognitive functioning, mood, energy, alertness, focus, performance, effects of aging, etc.); improve and/or maintain body composition; function as a therapeutic for one or more of the described conditions or disorders (e.g., treat neurological disorders); and/or combinations thereof. the stimulant component may include, for example but not limited to, adenosine receptor antagonists, phosphodiesterase inhibitors, epinephrine norepinephrine promoters, and/or xanthan alkaloids such as caffeine, theacrine, theobromine, theophylline, methylliberine, caffeine analogs, or combinations thereof.

In some implementations, the stimulant component may independently be capable of increasing ketone levels, or may increase ketone levels greater than merely the capability of each component individually (e.g., greater than an additive increase). For example, in some implementations, the composition may include caffeine in amounts of less than approximately 500 mg, from about 5 mg to about 500 mg, from about 10 mg to about 150 mg, from about 10 mg to about 50 mg. In some implementations, the composition may include theacrine, also known as 1,3,7,9-Tetramethyluric acid and/or TeaCrine® (commercially available as; from Compound Solutions, Calif., USA). In some implementations, the composition may include less than approximately 500 mg of theacrine.

In some implementations, the composition can include other carbon length ketones. For example, in some implementations, the composition may include one or more four carbon (C4) ketone bodies, or pharmaceutically acceptable free acid, salt, ester, or chelate thereof in some implementations, the C4 ketone bodies may be β-hydroxybutyrate or pharmaceutically acceptable free acid, salt, ester, or chelate thereof. In some implementations, (R)-β-hydroxybutyrate may be administered to manage glucose tolerance, reducing fasting glucose levels, and/or maintain and/or promote ketosis (e.g., maintain and/or increase blood ketone levels). As described in herein and in U.S. patent application Ser. Nos. 15/491,924 and 17/367,206, to Lowery et al. and entitled “Administration of B-hydroxybutyrate and Related Compounds in Humans”, which is incorporated by reference to the extent that the teachings do not conflict with the present disclosure. Also as described in herein and in U.S. Patent Application Nos. ______ filed Apr. 22, 2022 to Lowery et al. and entitled “Administration of (R)-β-hydroxybutyrate Salt Blend and Related Compounds in Humans”, which is incorporated by reference to the extent that the teachings do not conflict with the present disclosure.

In some embodiments, the composition may be delivered as a co-therapy or combination therapy, comprising administration of a therapeutically effective amount of a C5 ketone body, or pharmaceutically acceptable salt, ester, or chelate thereof, and other carbon length ketones, therapeutically effective amount means that amount of the combination of agents taken together so that the combined effect elicits the desired biological or medicinal response.

For example, in some implementations, the composition may include approximately 0.5 to approximately 50 g of C5 ketone bodies and 0.5 to 20 g of β-hydroxybutyrate (e.g., free BHB, free RBHB, sodium β-hydroxybutyrate, potassium β-hydroxybutyrate, calcium β-hydroxybutyrate, etc.). The combination of the C5 ketone bodies and the β-hydroxybutyrate may provide unexpected increase in blood ketone levels when compared to the components individually and/or at least partially enter the Krebs cycle.

The C4 ketone bodies may include racemic and/or enriched mixtures (e.g., one form may be predominant) of the different enantiomers (R/S or D/L) to produce a predetermined effect during administration. For example, the C4 ketones utilized in the methods and compositions may be provided in an isolated or purified form. Synthetic variants of the C5 ketones may be utilized, either individually or in combination with other synthetic C5 ketone variants, or in combination with non-synthetic variants. For example, in some embodiments, the composition may comprise a non-racemic mixture containing more than 50% and less than 100% by equivalents of the (R)-β-hydroxybutyrate enantiomer.

The C4 ketone bodies may include one or more C4 ketone body salts (e.g., sodium β-hydroxybutyrate, potassium β-hydroxybutyrate, calcium β-hydroxybutyrate, etc.). The cation associated with the C4 ketone body salts may be any appropriate cation such as sodium, potassium, magnesium, and calcium, and transition metals such as zinc, selenium, chromium, and iron, as well as other appropriate minerals, and/or combinations thereof.

In some implementations, other compounds, such as compounds capable of independently decreasing glucose levels, may be administered with β-hydroxybutyrate, such as berberine and/or associated metabolites (e.g., dihydroberberine and/or tetrahydroberberine). U.S. patent application Ser. No. 15/491,933 entitled “Administration of Dihydroberberine” to Lowery et al, filed Apr. 19, 2017, and U.S. Provisional Patent Application No. 62/324,794, entitled “Administration of Dihydroberberine” to Lowery et al, filed Apr. 19, 2016, describe dihydroberberine administration with ketone sensitizers such as β-hydroxybutyrate, and is hereby fully incorporated herein. U.S. patent application Ser. No. 17/341,312 entitled “Administration of Berberine Metabolites” to Lowery et al, filed Jun. 7, 2021, describe dihydroberberine administration with ketone sensitizers such as β-hydroxybutyrate, and is hereby fully incorporated herein.

In some implementations, a berberine, such as dihydroberberine, may cause reduce and/or maintain low fasting glucose. Administration of the composition with a berberine, such as dihydroberberine, may cause reduce and/or maintain low glucose levels. In some implementations, less than approximately 15 g of C5 ketone bodies may be administered with less than approximately 600 mg of dihydroberberine. One or more C5 ketone bodies, β-hydroxybutyrates, and/or other compounds described herein may be utilized as a ketone sensitizer with the dihydroberberine.

In some implementations, additional compounds may not be capable of independently increasing blood ketone levels and/or decreasing blood glucose levels (e.g., additives, flavorings, colorings, minerals, vitamins, binders, anti-caking agents, etc.).

For example, in some implementations, the composition may include or be administered with one or more other compounds such as, but not limited to, amino acids, amino acid metabolites, proteins (e.g., dairy proteins such as whey and/or casein; vegan proteins such as pea protein and/or pumpkin protein; egg protein; meat derived proteins; any other appropriate protein; and/or combinations thereof), vitamins, minerals, herbs and/or extracts of herbs, coconut milk (e.g., powder), flavorings, colorings, binders, electrolytes, tetrahydrobiopeterin, nucleic acids, alpha-ketoglutaric acid, alpha lipoic acid, nutritional co-factors, β-methyl-β-hydroxybutyrate, arginine alpha-ketoglutarate, (R)-alpha lipoic acid, thiamine, NAD+, NADH, riboflavin, FAD+, FADH, riboflavin-5-phosphate, niacin, nicotinic acid, niacinamide, inositol hexanicotinate, pyridoxine, pyridoxal, pyridoxamine, ascorbic acid and ascorbate salts, citric acid, malic acid, sodium benzoate, Pyridoxal-5-Phosphate, methylcobalamin, cyanocobalamin, adenosylcobalamin, hydroxycobalamin, pantothenic acid, pantetheine, potassium sorbate, acesulfame K, aspartame, sucralose, stevia, monk fruit extract, allulose, prebiotic fibers, XOS, GOS, MOS, IMO, LOS, xanthan gum and other organic gums/thickeners/suspension agents, and combinations thereof. In some implementations, the composition may include collagen peptides and/or hyaluronic acid.

For example, in some implementations, the composition may include or be administered with one or more other compounds such as, but not limited to, amino acids (e.g., leucine), caffeine, flavors, vitamins, minerals, herb extracts (e.g., extract of Uncaria tomentosa), and/or pharmaceutically acceptable binders (e.g., fluid and/or solid binders). The composition may include bone broth. In some implementations, the composition may include fatty acids, esters, or triglycerides (e.g., short chain, long chain, and/or medium chain). In some implementations, the composition may include 0.5 to 3 g of C5 ketone bodies, caffeine, and/or fatty acids, esters, and/or triglycerides (e.g., medium, short, and/or long chain).

For example, in some implementations, the composition may include or be administered with one or more other compounds such as, but not limited to NAD, ketone esters, Sweeteners (e.g., natural and/or synthetic), Flavors, Vitamins, CMED 100, AcAc, Caffeine, MCTs, SCFA, Creatine, Protein, Glucose, Glutamine, Electrolytes (e.g., additionally and/or from the salt of the C5 ketone bodies and/or β-hydroxybutyrate), Pyruvate, Lactate, and/or combinations thereof.

In some implementations, one or more additives may be included in the composition, such as flavorings (e.g., natural and/or artificial), vitamins, minerals, binders, pharmaceutically acceptable carriers (e.g., liquid and/or other types of carriers) and/or any other appropriate additive. The additives may alter flavor, color, and/or texture. The additives may increase palatability and/or facilitate inclusion in a delivery vehicle (e.g., tablet, food product, beverage product such as a drink mix, etc.). The additive may be any appropriate solid and/or liquid to which the compound is added. For example, an additive may include liquid carriers, such as water, milk(s), bone broth, and/or any other appropriate drinkable liquid. In some implementations, the (R)-β-hydroxybutyrate salt blend composition may include a pharmaceutically inert liquid carrier, such as water (e.g., tap water, filtered water, distilled water, etc.). The liquid carrier may include other drinkable liquids such as coconut water, watermelon water, electrolyte water, bone broth, and/or combinations thereof. The liquid carrier may include milks such as dairy milk, non-dairy milk, coconut milk, other milks, and/or combinations thereof. The liquid carrier may include an electrolyte solution, in some implementations. Use of a liquid carrier, such as bone broth may facilitate maintenance of ketosis or cause ketosis while providing satiety.

The composition may be administered in any appropriate delivery form (e.g., tablet; capsule; food products such as powdered products that can be mixed into food, mixed into beverages, and/or consumed directly; beverage product; etc.).

For example, the composition may be administered via any appropriate administration method. For example, the described compositions may be administered enterally and/or parenterally. In some implementations, the described composition may be administered via a tablet and/or capsule. The described composition may be provided in a powdered form that allows the described composition to be sprinkled on food, mixed with a liquid to provide a beverage, and/or directly administered. The described composition may be provided in gel form. The compounds in the composition may be mixed, coupled to each other, and/or provided separately. For example, the composition may include β-hydroxypentoate coupled to another compound (e.g., β-hydroxypentoate ester and/or amino acid). In some implementations, the first mixture and one or more other compounds may be provided separately (e.g., in pills, packets, etc.). An individual may sequentially and/or concurrently be administered (e.g., swallow pills) the β-hydroxypentoate and other compounds.

In various implementations, the composition may be delivered mixed in a powdered, liquid, gel, and/or other appropriate form. In some implementations, the composition may be administered via pills, tablets, capsules, other oral administration forms, intravenously, nasal sprays, sublingual tabs/strips, or topical delivery, rectal, other appropriate administration forms, and/or combinations thereof.

Administration can be locally (confined to an area of body) and/or systemically in the subject. It may be desirable to administer the C5 ketones, or pharmaceutical compositions comprising same, locally to the area in need of treatment. This method of administration may be achieved by, for example, and not by way of limitation, local infusion, topical application, by injection, by catheter, or by means of an implant (e.g., a porous membrane). Administration may also be orally, such as but not limited to, by way of pills, capsules, liquid, vapors, or powders.

In some implementations, the C5 ketones, or pharmaceutical composition containing them, can be delivered in a controlled release system. Such methods may include the use of a pump for administration (e.g., use of an intravenous drip).

In some implementations, if for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agents. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

In some implementations, if a packaging material is utilized to package the composition, it may be biologically inert or lack bioactivity, such as plastic polymers, silicone, etc. and may be processed internally by the subject without affecting the effectiveness of the components packaged and/or delivered therewith. Additionally, the compositions and components may be packaged with additional agents.

The composition may be administered according to any appropriate schedule (e.g., periodic dosages, dosages as user desires, etc.). The composition administration schedule may inhibit administration that elevates blood ketone levels too high, decreases blood glucose levels too low, and/or causes an individual to consume a dosage that substantially elevates the risk of adverse and/or side effects, in some implementations.

The schedule may be an administration protocol to cause weight loss and/or maintain a weight of an individual; elevate and/or maintain blood ketone levels; increase and/or maintain ketosis; and/or improve glucose tolerance (e.g., fasting glucose levels may be reduced and/or glucose metabolism may be improved), in some implementations. For example, the described compositions may be administered once a day, via an extended-release preparation, and/or multiple times a day (e.g., 1 to 5 times a day, 2 to 5 times a day, 3 to 5 times a day, etc.). The described composition may replace other pharmaceuticals or dietary supplements taken to promote weight loss, maintain a weight, promote ketosis, elevate blood ketone levels and/or be utilized in combination with one or more other pharmaceuticals or dietary supplements, as appropriate. The described composition may replace other pharmaceuticals or dietary supplements taken for improving glucose tolerance, such as metaformin, and/or be utilized in combination with one or more other pharmaceuticals or dietary supplements, as appropriate, in some implementations.

In some implementations, a first dose(s) may include at least one non-long-acting form of C5 ketone body, and a second dose(s) may include at least one long-acting form of C5 ketone body. The first dose(s) may be administered to cause a predetermined health impact and the second dose(s) may be administered to maintain the caused predetermined health impact. In some implementations, users may select the appropriate dose based on user preference and/or properties (e.g., a user on a ketogenic diet may chose the second dose since the user may already be in ketosis).

In some implementations, a kit may be provided with the composition. The composition may include one or more C5 ketone or pharmaceutically acceptable salt, ester, or chelate thereof, as described herein. The kits may further be used in the methods described herein. The kits may also include at least one reagent and/or instructions for their use. Also, the kit may include one or more containers filled with reagent(s) and/or one or more components of the disclosure. One or more container of the kits provided may also comprise a C5 ketone, preferably in a purified form. Also, one or more container of the kits provided may also comprise another carbon length ketone, preferably in a purified form. In some implementations, the various C5 ketones and other carbon length ketones may be provided in separate containers for mixing prior to use, or for individual ingestion/administration. The kits may also comprise a control composition, such as reagents that lack a ketone for use as a control reagent in experimentation. As it would be understood by those skilled in the art, detection or labeling methodologies can be used in the kits provided when utilized in an experimental or laboratory setting.

In some implementations, the kit may comprise a composition containing more than 0% and less than 100%, by weight, of at least one five carbon (C5) ketone body; and a pharmaceutically or dietetically acceptable carrier. The kit may additionally comprise a container in which the composition is placed. The container can be a carton, box, can, jar, bag, pouch, bottle, jug, and/or keg, as well as other containers suitable for holding the composition. The kit may additionally comprise a measuring device configured to hold therein a unit dose, or fraction thereof, of the composition. The measuring device can be a cup, scoop, syringe, dropper, spoon, and/or colonic irrigation device, as well as other suitable devices for measuring and/or dispensing a unit dose. In some embodiments, the unit dose contains from about 100 mg to about 25 g of the composition.

### EXAMPLES

### Example 1: BHP and BKP Effects on Subject Blood Glucose and Ketone Levels; and Subject Well-being, Energy, and Focus

Two (2) healthy, male subjects reported to the laboratory following a 12 hour overnight fast. Subjects were instructed to randomly consume either 10 grams of BHP or 10 grams BKP dissolved in 12 ounces of water. Subjects were then monitored for 150 minutes following drinking the mixture. Blood glucose, ketones, and visual analog scales (VAS) of well-being, energy, and focus were taken at 0, 30, 60, 90, 120, and 150 minutes. VAS scales consisted of a continuum of numbers from 1-10 with 1 being extremely low and 10 being extremely high.

Blood ketones were measured via finger prick using an Abbott Precision device which determines mMol of BHB. Blood glucose was measured using Abbott Precision glucose strips and was measured in mg/dL.

Results are summarized in the following tables.

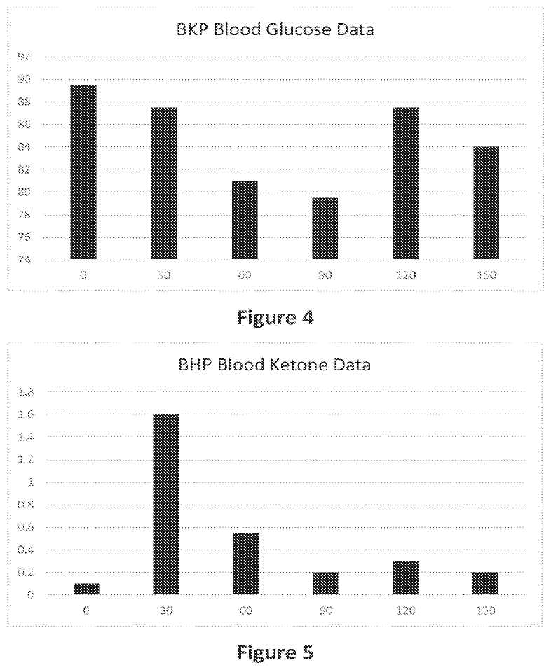

Table 1 shows blood glucose level following the administrations of 10 grams of BHP and BKP, recorded at 30-minute intervals. FIG. 4 shows a corresponding graph of blood glucose level following administration of BKP.

TABLE 1
Blood glucose levels (mg/dL) following BHP and BKP administration

| | 0 min. | 30 min. | 60 min. | 90 min. | 120 min. | 150 min. |
|---|---|---|---|---|---|---|
| Subject 1 | | | | | | |
| BHP | 84 | 85 | 75 | 79 | 86 | 91 |
| BKP | 88 | 84 | 79 | 83 | 85 | 87 |
| Subject 2 | | | | | | |
| BHP | 84 | 76 | 66 | 79 | 81 | 90 |
| BKP | 91 | 91 | 83 | 76 | 90 | 81 |
| Combined | | | | | | |
| BHP | 84 | 80.5 | 70.5 | 79 | 83.5 | 90.5 |
| BKP | 89.5 | 87.5 | 81 | 79.5 | 87.5 | 84 |

Table 2 shows blood ketone level following the administrations of 10 grams of BHP and BKP, recorded at 30-minute intervals. FIG. 5 shows a corresponding graph of blood ketone level following administration of BHP.

TABLE 2
Blood ketone levels (mMol) following BHP and BKP administration

| | 0 min. | 30 min. | 60 min. | 90 min. | 120 min. | 150 min. |
|---|---|---|---|---|---|---|
| Subject 1 | | | | | | |
| BHP | 0.1 | 1.8 | 0.7 | 0.2 | 0.2 | 0.2 |
| BKP | 0.1 | 0.1 | 0.2 | 0.1 | 0.1 | 0.1 |
| Subject 2 | | | | | | |
| BHP | 0.1 | 1.4 | 0.4 | 0.2 | 0.4 | 0.2 |
| BKP | 0.1 | 0.1 | 0.2 | 0.1 | 0.1 | 0.1 |
| Combined | | | | | | |
| BHP | 0.1 | 1.6 | 0.55 | 0.2 | 0.3 | 0.2 |
| BKP | 0.1 | 0.1 | 0.2 | 0.1 | 0.1 | 0.1 |

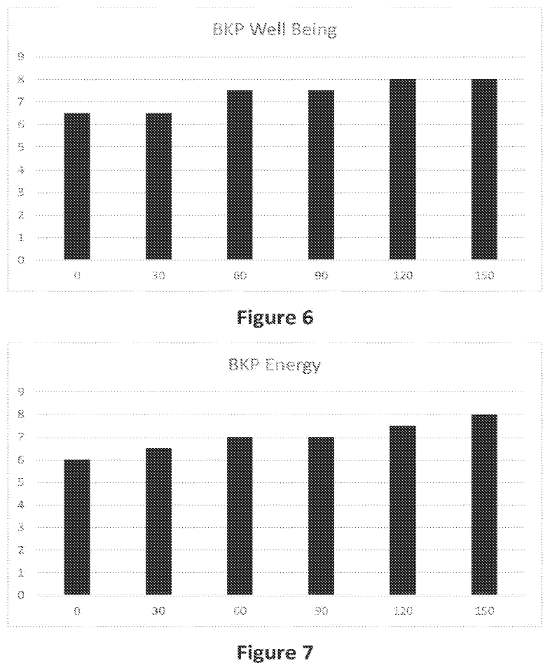

Table 3 shows visual analog scales (VAS) of perceived well-being following the administrations of 10 grams of BHP and BKP, recorded at 30-minute intervals. FIG. 6 shows a corresponding graph of VAS of perceived well-being following BKP administration.

TABLE 3
VAS of Well-Being following BHP and BKP administration

| | 0 min. | 30 min. | 60 min. | 90 min. | 120 min. | 150 min. |
|---|---|---|---|---|---|---|
| Subject 1 | | | | | | |
| BHP | 7 | 5 | 5 | 7 | 5 | 6 |
| BKP | 7 | 7 | 9 | 9 | 9 | 9 |
| Subject 2 | | | | | | |
| BHP | 5 | 5 | 4 | 5 | 4 | 4 |
| BKP | 6 | 6 | 6 | 6 | 7 | 7 |
| Combined | | | | | | |
| BHP | 6 | 5 | 4.5 | 6 | 4.5 | 5 |
| BKP | 6.5 | 6.5 | 7.5 | 7.5 | 8 | 8 |

Table 4 shows visual analog scales (VAS) of perceived energy following the administrations of 10 grams of BHP and BKP, recorded at 30-minute intervals. FIG. 7 shows a corresponding graph of VAS of perceived energy following BKP administration.

TABLE 4
VAS of Energy following BHP and BKP administration

| | 0 min. | 30 min. | 60 min. | 90 min. | 120 min. | 150 min. |
|---|---|---|---|---|---|---|
| Subject 1 | | | | | | |
| BHP | 5 | 4 | 5 | 7 | 5 | 7 |
| BKP | 7 | 7 | 7 | 8 | 8 | 9 |
| Subject 2 | | | | | | |
| BHP | 3 | 3 | 2 | 3 | 4 | 5 |
| BKP | 5 | 6 | 7 | 6 | 7 | 7 |
| Combined | | | | | | |
| BHP | 4 | 3.5 | 3.5 | 5 | 4.5 | 6 |
| BKP | 6 | 6.5 | 7 | 7 | 7.5 | 8 |

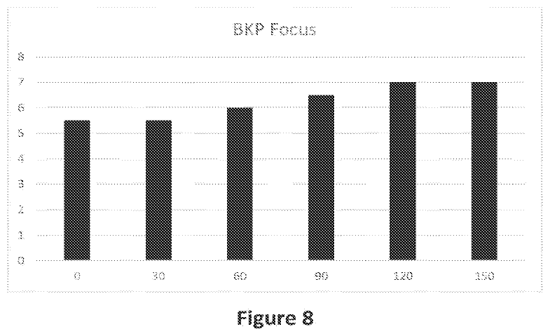

Table 5 shows visual analog scales (VAS) of perceived focus following the administrations of 10 grams of BHP and BKP, recorded at 30-minute intervals. FIG. 8 shows a corresponding graph of VAS of perceived focus following BKP administration.

TABLE 5
VAS of Focus following BHP and BKP administration

| | 0 min. | 30 min. | 60 min. | 90 min. | 120 min. | 150 min. |
|---|---|---|---|---|---|---|
| Subject 1 | | | | | | |
| BHP | 5 | 5 | 5 | 5 | 5 | 5 |
| BKP | 6 | 5 | 7 | 7 | 7 | 8 |
| Subject 2 | | | | | | |
| BHP | 2 | 3 | 3 | 3 | 5 | 5 |
| BKP | 5 | 6 | 5 | 6 | 7 | 6 |
| Combined | | | | | | |
| BHP | 3.5 | 4 | 3 | 3 | 5 | 5 |
| BKP | 5.5 | 5.5 | 6 | 6.5 | 7 | 7 |

### Example 2: C5 Ketone Administration as a Method to Lower Exertion During Exercise

The purpose of this experiment was to see if C5 ketones could lower exertion during moderate and high intensity cycling exercise. Five (5) college aged males were given a placebo or BKP (10 grams) prior to and during 2-minute low, moderate and high intensity cycling bouts. BKP is able to lower perceived exertion at both moderate and high intensity exercise compared to a placebo. See Table 6, which shows the Borg Rating of Perceived Exertion (RPE), which is a way of measuring physical activity intensity level. Perceived exertion is how hard one feels like the body is working.

TABLE 6
Rating of Perceived Exertion (RPE)

| | | Low | Moderate | High | 30 min.- |
|---|---|---|---|---|---|
| | Baseline | Intensity | Intensity | Intensity | Post |
| BKP | 7 ± 1 | 9 ± 1 | 13 ± 2 | 17 ± 2 | 8 ± 1 |
| Placebo | 7 ± 1 | 9 ± 1 | 14 ± 1 | 18 ± 1 | 9 ± 1 |

The results of these findings show that C5 ketone bodies lower blood glucose, increase blood ketone levels, and increase visual analog scales of well-being, focus, and energy in otherwise healthy males. The disclosure further shows that C5 ketones are able to lower perceived exertion at both moderate and high intensity exercise. This disclosure has significant implications ranging from therapeutic effects to cognitive and athletic performance in a wide array of populations.

Without further elaboration, it is believed that one skilled in the art can, using the preceding description, utilize the present invention to its fullest extent. The examples are offered by way of illustration, not by way of limitation. While specific examples have been provided, the above description is illustrative and not restrictive. Anyone or more of the features of the previously described implementations can be combined in any manner with one or more features of any other implementations in the present invention. Furthermore, many variations of the invention will become apparent to those skilled in the art upon review of the specification.

In various implementations C5 ketone bodies are described as included in a composition; administered in an amount, form, and/or schedule; and/or being in a particular form (e.g., complexed and/or coupled). The composition may be utilized in the various described implementations of C5 ketone bodies in the same or higher amounts as the described C5 ketone bodies, as appropriate.

The compounds and compositions of the invention may also be utilized in pharmaceutically acceptable compositions in the methods provided herein. It would also be understood by a skilled artisan how to use the compositions described herein for therapeutic purposes without undue experimentation based on the teachings provided throughout the specification.

The amount of active ingredients or pharmaceutical compounds of the invention which will be effective in the treatment, inhibition and/or prevention of the diseases and/or conditions described herein can be determined by standard clinical techniques. Additionally, in vitro assays may be employed to help identify optimal dosage ranges. The precise dose to be utilized will also depend on the route of administration, and the seriousness of the disease or condition, and should also be decided according to the sound medical judgment of the clinician and each patient's individual circumstances. The specific therapeutically effective dosage level for any particular patient will depend upon a variety of factors including: the type and degree of the response to be achieved; the specific composition and other agent(s), if any, employed; the age, weight, general health, sex and diet of the patient; the time of administration, route of administration, and rate of excretion of the composition; the duration of the treatment; other drugs used in combination or coincidental with the composition; and any other factors well known in the medical arts. Effective dosages may also be extrapolated from dose-response curves derived from in vitro or animal model testing systems.

Several aspects of the disclosure are described with reference to examples for illustrative purposes only. It should be understood that numerous specific details, relationships, and methods are set forth to provide a full understanding of the disclosure. One having ordinary skill in the relevant art, however, will readily recognize that the aspects described can be practiced without one or more of the specific details or practiced with other methods, protocols, reagents, and animals. The aspects described are not limited by the illustrated ordering of acts or events, as some acts may occur in different orders and/or concurrently with other acts or events. Furthermore, not all illustrated acts, steps or events are required to implement a methodology in accordance with the aspects described. Many of the techniques and procedures described, or referenced herein, are well understood, and commonly employed using conventional methodology by those skilled in the art.

Although the present disclosure has been described in detail, it should be understood that various changes, substitutions, and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A composition comprising:
more than 0% and less than 100%, by weight, of at least one five carbon (C5) ketone body; and
a pharmaceutically or dietetically acceptable carrier.

2. The composition of claim 1, wherein the C5 ketone body is selected from:
β-hydroxypentanoate having the chemical structure: 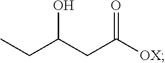
β-ketopentanoate having the following chemical structure: 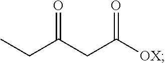
and
combinations thereof;
wherein X is a hydrogen, a metal ion, an amino cation, an amino acid cation, an alkane, an alkenyl, or an aryl.

3. The composition of claim 1, wherein the C5 ketone body is selected from the group consisting of:
(R)-β-hydroxypentanoate;
(S)-β-hydroxypentanoate;
(R)-β-ketopentanoate;
(S)-β-ketopentanoate; and
combinations thereof.

4. The composition of claim 3, wherein administration of (S) enantiomers results in at least one of:
increased endogenous production of (R)-β-hydroxypentanoate;
increased endogenous production of (R)-β-ketopentanoate;
endogenous conversion of the (S)-β-hydroxypentanoate into (R)-β-hydroxypentanoate;
endogenous conversion of the (S)-β-ketopentanoate into (R)-β-ketopentanoate;
endogenous conversion of the (S)-β-hydroxypentanoate into fatty acids and sterols;
endogenous conversion of the (S)-β-ketopentanoate into fatty acids and sterols;
metabolism the (S)-β-hydroxypentanoate independent of conversion to (R)-β-hydroxypentanoate;
metabolism the (S)-β-ketopentanoate independent of conversion to (R)-β-ketopentanoate; and
prolonged ketosis.

5. The composition of claim 3, wherein the composition further comprises a non-racemic mixture of β-hydroxypentanoate containing:
more than 50% and less than 100% by equivalents of (R)-β-hydroxypentanoate; and
more than 0% and less than 50% by equivalents of (S)-β-hydroxypentanoate.

6. The composition of claim 5, wherein the enantiomeric ratio of (R)-β-hydroxypentanoate to (S)-β-hydroxypentanoate is at least 2:1.

7. The composition of claim 3, wherein the composition further comprises a non-racemic mixture containing:
more than 50% and less than 100% by equivalents of (S)-β-hydroxypentanoate; and
more than 0% and less than 50% by equivalents of (R)-β-hydroxypentanoate.

8. The composition of claim 7, wherein the enantiomeric ratio of (S)-β-hydroxypentanoate to (R)-β-hydroxypentanoate is at least 2:1.

9. The composition of claim 3, wherein the composition further comprises a non-racemic mixture of β-ketopentanoate containing:
more than 50% and less than 100% by equivalents of (R)-β-ketopentanoate; and
more than 0% and less than 50% by equivalents of (S)-β-ketopentanoate.

10. The composition of claim 9, wherein the enantiomeric ratio of (R)-β-ketopentanoate to (S)-β-ketopentanoate is at least 2:1.

11. The composition of claim 3, wherein the composition further comprises a non-racemic mixture of β-ketopentanoate containing:
more than 50% and less than 100% by equivalents of (S)-β-ketopentanoate; and
more than 0% and less than 50% by equivalents of (R)-β-ketopentanoate.

12. The composition of claim 11, wherein the enantiomeric ratio of (S)-β-ketopentanoate to (R)-β-ketopentanoate is at least 2:1.

13. The composition of claim 3, wherein the C5 ketone body is further selected from the group consisting of:
one or more salt of the C5 ketone body;
one or more amino acid salt of the C5 ketone body;
one or more ester of the C5 ketone body;
one or more acid of the C5 ketone body;
one or more polymer of the C5 ketone body; and
combinations thereof.

14. The composition of claim 13, wherein the salt of the C5 ketone body is selected from the group consisting of sodium, potassium, calcium, magnesium, one or more transition metal, and combinations thereof.

15. The composition of claim 14, wherein the salt is a combination of at least two salts, a combination of at least three salts, or a combination of at least four salts.

16. The composition of claim 14, wherein the transition metal is iron or zinc.

17. The composition of claim 14, wherein the amino acid salt includes at least one cationic amino acid or amino acid metabolite.

18. The composition of claim 14, wherein the amino acid salt is selected from salts of arginine, lysine, leucine, iso-leucine, histidine, ornithine, citrulline, glutamine, or creatine.

19. The composition of claim 14, wherein the ester of the C5 ketone body comprises at least one of monoester of ethanol, monoester of 1-propanol, monoester of 1,3-propanediol, di-ester of 1,3-propanediol, mono- or di-ester of S-1,3-butanediol, mono- or di-ester of (R)-1,3-butanediol, mono- or di-ester of (R)-1,3-butanediol, or mono-, di-, or tri-ester of glycerin.

20. The composition of claim 14, wherein the acid of the C5 ketone body is selected from the group consisting of:
β-hydroxypentanoic acid;
β-ketopentanoic acid; and
combinations thereof.

21. The composition of claim 1, further comprising:
at least one citrate salt selected from sodium citrate, potassium citrate, calcium citrate, and magnesium citrate.

22. A method for aiding in at least one of weight loss or fat loss in an individual, the method comprising:
administering a composition to the individual, wherein the composition comprises:
more than 0% and less than 100%, by weight, of at least one five carbon (C5) ketone body; and
a pharmaceutically or dietetically acceptable carrier;
wherein administration of the composition aids in at least one of weight loss and/or fat loss in the subject individual.

23. The method of claim 22, wherein the C5 ketone body comprises a non-racemic mixture of β-hydroxypentanoate containing:
more than 50% and less than 100% by equivalents of (R)-β-hydroxypentanoate; and
more than 0% and less than 50% by equivalents of (S)-β-hydroxypentanoate.

24. The method of claim 22, wherein the C5 ketone body comprises a non-racemic mixture of β-ketopentanoate containing:
more than 50% and less than 100% by equivalents of (S)-β-ketopentanoate; and
more than 0% and less than 50% by equivalents of (R)-β-ketopentanoate.

25. A method for increasing blood ketone levels in an individual, the method comprising:
administering a composition to the individual, wherein the composition comprises:
more than 0% and less than 100%, by weight, of at least one five carbon (C5) ketone body; and
a pharmaceutically or dietetically acceptable carrier;
wherein administration of the composition increases blood ketone levels in an individual.

26. The method of claim 25, wherein the C5 ketone body comprises a non-racemic mixture of β-hydroxypentanoate containing:
more than 50% and less than 100% by equivalents of (R)-β-hydroxypentanoate; and
more than 0% and less than 50% by equivalents of (S)-β-hydroxypentanoate.

27. The method of claim 25, wherein the C5 ketone body comprises a non-racemic mixture of β-ketopentanoate containing:
more than 50% and less than 100% by equivalents of (S)-β-ketopentanoate; and
more than 0% and less than 50% by equivalents of (R)-β-ketopentanoate.

28. A method for managing cognitive function in an individual, the method comprising:
administering a composition to the individual, wherein the composition comprises:
more than 0% and less than 100%, by weight, of at least one five carbon (C5) ketone body; and
a pharmaceutically or dietetically acceptable carrier;
wherein administration of the composition as in at least one of restoring cognitive function, increasing cognitive function and/or slowing cognitive decline in an individual.

29. The method of claim 28, wherein the C5 ketone body comprises a non-racemic mixture of β-hydroxypentanoate containing:
more than 50% and less than 100% by equivalents of (R)-β-hydroxypentanoate; and
more than 0% and less than 50% by equivalents of (S)-β-hydroxypentanoate.

30. The method of claim 28, wherein the C5 ketone body comprises a non-racemic mixture of β-ketopentanoate containing:
more than 50% and less than 100% by equivalents of (S)-β-ketopentanoate; and
more than 0% and less than 50% by equivalents of (R)-β-ketopentanoate.
